(19) United States
(12) Patent Application Publication
(10) Pub. No.: US 2012/0082656 A1
(43) Pub. Date: Apr. 5, 2012

(54) COMPOSITION COMPRISING THE PURIFIED EXTRACT OF BEE VENOM FOR PREVENTING AND TREATING DEGENERATIVE BRAIN DISEASE

(76) Inventors: Seong Tae Yoon, Seoul (KR); Dae Hee Shin, Seoul (KR); Bang Ho Lim, Seoul (KR); Jae Young Kim, Gyeonggi-do (KR); Jae Seok Choi, Daejeon-si (KR)

(21) Appl. No.: 13/265,777

(22) PCT Filed: Dec. 4, 2009
(86) PCT No.: PCT/KR2009/007234

§ 371 Date: Dec. 21, 2011

(30) Foreign Application Priority Data
May 22, 2009 (KR) 10-2009-0044995

(51) Int. Cl. A61K 38/46; A61P 25/16; A61P 25/28

(52) U.S. Cl. 424/94.6

(57) ABSTRACT

The present invention relates to a composition comprising the purified extract of bee venom for preventing and treating degenerative brain diseases and the use thereby. The purified extract of bee venom shows potent inhibitory effect on microglial cell activation and abnormal circling behavior using by animal model of degenerative brain disease as well as potent cell-protective activity therefore, it can be useful in treating and preventing the degenerative brain disease as a medicament.

### TECHNICAL FIELD

The present invention relates to a composition comprising the purified extract of bee venom for preventing and treating degenerative brain diseases and the use thereby.

### BACKGROUND ART

In the twentieth century, as the average life span of human has been increasing with the rapid development of life science and medicine, new social problems including increased population ratio of older people are coming to the front, especially, the geriatric neuronal diseases such as stroke, Alzheimer's disease (AD), Parkinson's disease (PD) etc., which are fatal functional disorder of neuronal system, have been increased.

Especially, Parkinson's disease is caused by the neuronal death of substantia nigra pars compacta in brain and is frequently occurring neuronal disease. In particular, in-diopathic Parkinson's disease predominates over 80% among the people suffering from Parkinson's disease and is an etiological cause by severe disorder in the older people (Ennio Esposito et al., Non-steroidal anti-inflammatory drugs in Parkinson's disease, Exp. Neurol., 205, pp 295-312, 2007).

Due to the recent aging society, the number of patient suffering from Parkinson's disease has increased till now and the drug market in 2013 is supposed to be increased by 139%, i.e., 2,370 million dollar (USD) compared by that in 2006.

At the present, the drug therapy using by levodopa, a representative drug for treating Parkinson's disease, has been conventionally available till now however it has several problems for example, the need to inconsistent administration, the therapeutic limit to fundamental treatment etc or disadvantages for example, a neuronal damage caused by dopamine administration, the reduced drug potency due to drug intolerance, post-treatment complication such as the disorder of involuntary movement, the aggravated movement disorder, the other adverse action associated from long-term drug therapy etc (Judith L. Miller, Parkinson's disease primer, Geriatric Nursing, 23(2), pp 69-73, 2002; Fabio Danisi, Parkinson's disease-Therapeutic strategies to improve patient function and quality of life, Geriatrics, 57(3), pp 46-50, 2002).

Accordingly, there have been still needed to develop new anti-Parkinson's drug and recent development and research has been focused in the aspect of various mechanisms, for example, dopaminergics, dopamine agonists, catechol-O-methyltransferase (COMT) inhibitors, monoamine oxidase inhibitors, adenosine a2a receptor antagonists; neuroprotective drugs such as dopamine receptor agonist, NMDA receptor agonist, anti-oxidant, NSAIDs, nicotinic acetylcholine receptor agonists, neurotrophic factor etc; and other therapeutic methods such as Cell/Gene therapy, stem cell differentiation, transplantation etc (Jun Takahashi, Stem cell therapy for Parkinson's disease, Expert Rev. Neurother., 7(6), pp 667-675, 2007).

Bee Venom, a bee sting exuded from the abdomen of various bee such as honey bee, bumble bee, sweat bee etc, shows weak aromatic property having the pH of 5.2 and bitter taste. It has been reported that it blocks the inflammatory pathway induced by increased factors such as NO, PGE2, TNF-a and the expression of inflammatory genes, resulting in potent anti-inflammatory activity which could treat various inflammatory pains such as neuralgia, rheumatism, back pain etc (Dong Ju Son et al., Therapeutic application of anti-arthritis, pain-releasing, and anti-cancer effect of bee venom and its constituent compounds, Pharmacol. Therapeutics, 115, pp 246-270, 2007). It has been reported that about 30% dried product and about 75% proteins such as melittin, apamin, phospholipase, adolapine, hyaluronidase, histidine and histamine etc remain after the evaporation at room temperature (Meier J. et al., Clinical toxicology of animal venoms and poisons, CRC Press Inc., 1995).

However, there has been not reported or disclosed about the therapeutic effect or improving effect for brain degenerative disease of the purified bee venom extract in any of above cited literatures, the disclosures of which are incorporated herein by reference.

To investigate the treating effect of bee venom on brain degenerative disease, the inventors of the present invention have intensively carried out several animal model tests such as neuro-protective activity, the inhibitory effect of microglial cell activation and abnormal spinning motor using by animal model of degenerative brain disease, and human neuroblastoma SH-SY5Y cell line together with human clinical test, and finally completed present invention by confirming that the purified extract of bee venom shows potent inhibitory effect on microglial cell activation and abnormal circling behavior using by animal model of degenerative brain disease as well as potent cell-protective activity.

These and other objects of the present invention will become apparent from the detailed disclosure of the present invention provided hereinafter.

### DISCLOSURE OF INVENTION

### Technical Problem

The present invention provides a pharmaceutical composition comprising the purified extract of bee venom as an active ingredient in an effective amount for preventing and treating degenerative brain diseases by protecting neuronal cell.

The present invention also provides a use of above extract for the preparation of pharmaceutical composition to treat and prevent degenerative brain disease by protecting neuronal cell in mammal or human.

### Solution to Problem

Accordingly, it is an object of the present invention to provide a pharmaceutical composition comprising the purified extract of bee venom as an active ingredient for the treatment and prevention of degenerative brain disease by protecting neuronal cell.

The term “purified extract” disclosed herein comprises the crude purified extract and the purified extract of bee venom such as honey bee, bumble bee, sweat bee etc, preferably, the purified extract of honey bee venom.

Specifically, the above-described crude purified extract of bee venom may comprise the extract prepared by the procedure comprising the steps of: dissolving dried bee venom collected from honey bee in water, lower alcohols such as methanol, ethanol, or butanol, preferably in water, subjecting the solution to filtration to remove impurities from the solution, and drying the filtrates with lyophilization to obtain the inventive crude purified extract of bee venom.

The above-described purified extract of bee venom may comprise the extract prepared by the procedure comprising the steps of; dissolving the dried crude purified extract of bee venom prepared in the above-step in water, lower alcohols such as methanol, ethanol, or butanol, preferably in water, subjecting the solution to at least one purification process selected from salting out method, solvent precipitation method, and dialysis membrane filtration in order to performing centrifugation or dialysis, and drying the filtrates with lyophilization to obtain the inventive purified extract of bee venom.

Preferably, the purified extract of bee venom may comprise the extract prepared by the procedure comprising the steps of; dissolving dried bee venom collected from honey bee in water, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to dialysis membrane filtration using by membrane dialysis to collect the purified extract present in the membrane and drying the filtrates with lyophilization at 3^{rd} step to obtain the inventive purified extract of bee venom having more potent pharmacological effect than the other extract of bee venom.

The inventive purified extract of bee venom having more potent pharmacological effect, may comprise melittin in an amount of ranging from about 30% to 90% (w/w %), preferably, about 35% to 80% (w/w %), more preferably, about 40% to 60% (w/w %), as an active ingredient.

It is an object of the present invention to provide a use of the purified extract of bee venom for the preparation of therapeutic agent for the treatment and prevention of degenerative brain disease by protecting neuronal cell in mammal including human.

It is an object of the present invention to provide a method of treating or preventing degenerative brain disease by protecting neuronal cell in mammal including human comprising administering an effective amount of the purified extract of bee venom, together with a pharmaceutically acceptable carrier thereof to said mammal.

The term “degenerative brain disease” disclosed herein comprises Alzheimer type dementia, cerebrovascular type dementia, pick's disease, Creutzfeldt-jakob's disease, dementia caused by cephalic damage, Parkinson's disease, and so on, preferably, Parkinson's disease, more preferably, Parkinson's disease caused by the hyper-activation of microglial cell.

In detail, the inventive purified extract of bee venom of the present invention may be prepared as follows:

The purified extract of bee venom may be prepared by the procedure comprising the steps of; dissolving dried bee venom collected from honey bee in water, lower alcohols such as methanol, ethanol, or butanol, preferably in water, filtrating to remove impurities and drying with lyophilization to obtain the crude purified extract of bee venom (designated as “HP-1” hereinafter) at 1^{st} step; dissolving the crude purified extract prepared in step 1 in water, lower alcohols such as methanol, ethanol, or butanol, preferably in water, subjecting the solution to at least one purification process selected from salting out method, solvent precipitation method, and dialysis membrane filtration in order to performing centrifugation or dialysis, and drying the filtrates with lyophilization at 2^{nd} step to obtain the inventive purified extract of bee venom.

In the 1^{st} preferred embodiment of the present invention, the present invention also provides a method for preparing the purified extract of bee venom prepared by the procedure comprising the steps of; dissolving dried bee venom collected from honey bee in water, lower alcohols such as methanol, ethanol, or butanol, preferably in water, filtrating to remove impurities and drying with lyophilization to obtain the crude purified extract of bee venom comprising 10.2% phospholipase, 40.5% melittin, 3.8% apamine, 1.6% histamine, 1.1% dopamine and 0.3% adrenaline (designated as “HP-01” hereinafter).

In the 2nd preferred embodiment of the present invention, the present invention also provides a method for preparing the purified extract of bee venom comprising the steps of; dissolving dried bee venom collected from honey bee in water, lower alcohols such as methanol, ethanol, or butanol, preferably in water, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to gel filtration chromatography and then protein dialysis membrane filtration using by membrane dialysis to perform salting out process at 3rd step; collecting the purified extract present in the membrane and drying the filtrates with lyophilization at 4th step to obtain the inventive purified extract of bee venom comprising 12.4% phospholipase, 48.4% melittin, 4.3% apamine, 0.9% histamine, 1.4% dopamine and 0.4% adrenaline (designated as “HP-01G” hereinafter).

In the 3rd preferred embodiment of the present invention, the present invention also provides a method for preparing the purified extract of bee venom prepared by the procedure comprising the steps of; dissolving dried bee venom collected from honey bee in water, lower alcohols such as methanol, ethanol, or butanol, preferably in water, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to salting-in process and then salt-out process using by using by salt such as ammonium sulfate to perform salting out process at 3rd step; centrifuging and lyophilizing the solution to obtain the supernatant at 4th step to obtain the inventive purified extract of bee venom in supernatant comprising <0.1% phospholipase, <0.1% melittin, <0.1% apamine, 8.4% histamine, 3.1% dopamine and 1.2% adrenaline (designated as “HP-01AL” hereinafter) and that in precipitant comprising 13.4% phospholipase, 53.6% melittin, 5.1% apamine, <0.1% histamine, <0.1% dopamine and <0.1% adrenaline (designated as “HP-01AP” hereinafter).

In the 4th preferred embodiment of the present invention, the present invention also provides a method for preparing the purified extract of bee venom prepared by the procedure comprising the steps of; dissolving dried bee venom collected from honey bee in water, lower alcohols such as methanol, ethanol, or butanol, preferably in water, filtrating to remove impurities and drying with lyophilization to obtain the crude purified extract of bee venom at 1^{st} step; dissolving the crude purified extract prepared in step 1 in the precipitation solvent selected from water, lower alcohols such as methanol, ethanol, or butanol or the mixture thereof, preferably, 50-90% ethanol, more preferably, 60-80% ethanol, to occur precipitation at 2^{nd} step; subjecting the solution to centrifugation and lyophilizing the solution to obtain the supernatant at 3rd step to obtain the inventive purified extract of bee venom in supernatant comprising <0.1% phospholipase, <0.1% melittin, <0.1% apamine, 12.1% histamine, 6.4% dopamine and 2.2% adrenaline (designated as “HP-01SL” hereinafter) and that in precipitant comprising 7.8% phospholipase, 56.4% melittin, 5.8% apamine, <0.1% histamine, <0.1% dopamine and <0.1% adrenaline (designated as “HP-01SP” hereinafter).

In the 5th preferred embodiment of the present invention, the present invention also provides a method for preparing the purified extract of bee venom prepared by the procedure comprising the steps of; dissolving dried bee venom collected from honey bee in water, lower alcohols such as methanol, ethanol, or butanol, preferably in water, filtrating to remove impurities and drying with lyophilization to obtain the crude purified extract of bee venom at 1^{st} step; dissolving the crude purified extract prepared in step 1 in the precipitation solvent selected from water, lower alcohols such as methanol, ethanol, or butanol or the mixture thereof, preferably, water, at 2^{nd} step; subjecting the solution to ultra-centrifugation using by ultra-centrifuges quipped with 50 kda membrane filter to obtain the purified extract of bee venom having high molecular weight of more than 50 kDa comprising 76.2% phospholipase, <0.1% melittin, <0.1% apamine, <0.1% histamine <0.1% dopamine and <0.1% adrenaline (designated as “HP-02A50” hereinafter) at 3rd step; centrifuging the fraction having low molecular weight of less than 50 kDa again using by ultra-centrifuges quipped with 10 kda membrane filter to obtain the purified extract of bee venom having higher molecular weight ranging from 10 kDa to 50 kDa comprising <0.1% phospholipase, 43.2% melittin, 6.2% apamine, <0.1% histamine <0.1% dopamine and <0.1% adrenaline (designated as “HP-03” hereinafter) and that having lower molecular less than 10 kDa comprising <0.1% phospholipase, <0.1% melittin, <0.1% apamine, 12.8% histamine 8.1% dopamine and 1.3% adrenaline (designated as “HP-04” hereinafter).

In the 6th preferred embodiment of the present invention, the present invention also provides a method for preparing the purified extract of bee venom prepared by the procedure comprising the steps of; dissolving dried bee venom collected from honey bee in water, lower alcohols such as methanol, ethanol, or butanol, preferably in water, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to dialysis process using by protein dialysis membrane at 3rd step; collecting the solution within the membrane and lyophilizing the solution to obtain the inventive purified extract of bee venom comprising 10.2% phospholipase, 40.5% melittin, 3.8% apamine, 1.6% histamine, 3.1% dopamine and 0.3% adrenaline (designated as “HP-05” hereinafter).

Therefore, the present invention also provides the above-described methods in order to obtain the inventive purified extract of bee venom and the purified extract of bee venom prepared by the above-described methods.

The inventive purified extract of bee venom having more potent pharmacological effect, may comprise phospholipase in an amount of ranging from about 1% to 80% (w/w %), preferably, about 3% to 50% (w/w %), more preferably, about 5% to 20% (w/w %); melittin in an amount of ranging from about 30% to 90% (w/w %), preferably, about 35% to 80% (w/w %), more preferably, about 40% to 60% (w/w %); and apamine in an amount of ranging from about 0.1% to 30% (w/w %), preferably, about 0.5% to 15% (w/w %), more preferably, about 1.0% to 10% (w/w %), as an active ingredient.

Through several animal model tests such as neuro-protective activity, the inhibitory effect of microglial cell activation and abnormal spinning motor using by animal model of degenerative brain disease, and human neuroblastoma SH-SY5Y cell line together with human clinical test, the purified extract of bee venom shows potent inhibitory effect on microglial cell activation and abnormal circling behavior using by animal model of degenerative brain disease as well as potent cell-protective activity therefore, it can be useful in treating and preventing the degenerative brain disease as a medicament.

The pharmaceutical composition of the present invention can contain about 0.01˜50% by weight of the above extract based on the total weight of the composition.

In accordance with another aspect of the present invention, there is provided a pharmaceutical composition comprising the purified extract of bee venom prepared by the above-described preparation methods an active ingredient for the treatment and prevention of degenerative brain disease by protecting neuronal cell.

It is another of the present invention to a use of the purified extract of bee venom prepared by the above-described preparation methods for the preparation of therapeutic agent for the treatment and prevention of degenerative brain disease by protecting neuronal cell in mammal including human.

It is an object of the present invention to provide a method of treating or preventing degenerative brain disease by protecting neuronal cell in mammal including human comprising administering an effective amount of the purified extract of bee venom prepared by the above-described preparation methods, together with a pharmaceutically acceptable carrier thereof to said mammal.

The inventive composition for treating and preventing degenerative brain disease by protecting neuronal cell may comprises the above extracts as 0.01˜50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

Pharmaceutical formulations containing present composition may be prepared in any form, for example, oral dosage form such as lyophilized preparation, powder, granule, tablet, capsule, soft capsule, elixirs pill, sachet etc as a solid oral formulation; suspension, solution, emulsion, syrup, aqueous medicine etc as a liquid oral formulation; topical preparation such as cream, ointment, lotion, gel, balm, patch, paste, spray solution, aerosol and the like; or parenteral dosage forms, for example, suppositories or injectable preparation such as sterilized solution, suspension, lyophilized preparation, non-aqueous type injection, or aqueous type injection, preferably, sterilized injectable preparation.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include solvent, additive, diluents, buffer, isotonic agent, stabilizer, anti-oxidant, pain-reliever, emulsifier, fillers, anti-agglutinating agents, lubricating agents, wetting agents, flavoring agents, preservatives etc. Specifically, said solvent, additive, or diluents includes sterilized distilled water, physiological saline solution, pH controller, albumin, sodium chloride, mannitol, Ringer's solution, glucose etc. The solid oral formulation such as powder, granule, tablet, capsule, soft capsule, elixirs pill, sachet etc may be prepared by mixing the inventive extract with at least one adjuvant, for example, starch, calcium carbonate, sucrose, lactose, gelatin etc, if necessary, lubricants such as magnesium stearate, talc etc as a additional additive to be formulated. The liquid oral formulation such as suspension, solution, emulsion, syrup, aqueous medicine etc may be prepared by mixing the inventive extract with at least one adjuvant, for example, wetting agent, flavoring agent, sweetener, preservative, other than common diluents such as water or liquid paraffin to be formulated. As the parenteral dosage forms, for example, injectable preparation such as sterilized solution, suspension, lyophilized preparation, non-aqueous type injection, or aqueous type injection, may use propylene glycol, polyethylene glycol, vegetable oil such as olive oil, injectable ester such as ethyl olate etc as a base; and suppositories may use whitepsol, macrogol, tween 61, cacao oil, lauric oil, glycerol-gelatin etc as a base in the present invention.

The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging from 1 microgram to 5 mg/day, preferably, 8 microgram to 2 mg/day, more preferably, 16 microgram to 1 mg/day of the inventive extract of the present invention. The dose may be administered in single or divided into several times per day; periodically, for example, once for a period ranging from 2 days to one week, but are not intended to limit thereto. The scope of present invention may include all the modification, or change in terms of any amount and number of dosage, and any administration pathway which can be conceivable by the artisan in the art. In terms of composition, the amount of inventive extract may be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Inventive extract of the present invention have no toxicity and adverse effect therefore; they can be used with safe.

It will be apparent to those skilled in the art that various modifications and variations can be made in the compositions, use and preparations of the present invention without departing from the spirit or scope of the invention.

### Advantageous Effects of Invention

Inventive extract of the present invention have no toxicity and adverse effect therefore; they can be used with safe.

It will be apparent to those skilled in the art that various modifications and variations can be made in the compositions, use and preparations of the present invention without departing from the spirit or scope of the invention.

### BRIEF DESCRIPTION OF DRAWINGS

The above and other objects, features and other advantages of the present invention will more clearly understood from the following detailed description taken in conjunction with the accompanying drawings, in which;

FIG. 1 shows the comparison of the HPLC chromatogram of HP-01, HP-05 and melittin standard;

FIG. 2 shows the cell survival rate of SH-SY5Y cells in control group, MPP+ group, and various concentrations of HP-01, HP-05 groups;

FIG. 3 represents the comparison of neuro-protective activity of control group, MPTP group, HP-01 and HP-05 in MPTP-induced Parkinson's disease animal model (A: the photograph of striatum (ST) and substantia nigra (SN)/B: the comparison of the number of dopaminergic neuron at SN/C: the comparison of TH-staining intensity at ST);

FIG. 4 represents the comparison of protective effect of control group, MPTP group, HP-01 and HP-05 on microglia cells in MPTP-induced Parkinson's disease animal model (arrow: CD11B positive cells);

FIG. 5 presents the comparison of inhibitory effect of control group, 6-OHDA group, HP-01 and HP-05 on abnormal circling behavior in 6-OHDA-induced Parkinson's disease animal model;

FIG. 6 depicts the comparison of neuro-protective effect of control group, 6-OHDA group, HP-01 and HP-05 in 6-OHDA-induced Parkinson's disease animal model.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### Mode for the Invention

The following Reference Example, Examples and Experimental Examples are intended to further illustrate the present invention without limiting its scope.

### Example 1

### Preparation of the Crude Purified Extract of Bee Venom

10.0 g of dried bee venom collected from honey bee was dissolved in waster and the impurities were removed by filtration using by syringe filter (Minisart RC 15, 0.20 microm, Sartorius Co. Germany). The filtrate was dried with lyophilizer (FDCF-12012, Operon Co. Korea) to obtain 9.76 g of dried crude purified extract of bee venom (designated as “HP-01” hereinafter). The dried powder was used in following experiments as a test sample.

### Example 2

### Preparation of Purified Extract of Bee Venom Using by Gel Filtration Chromatography

100 mg of the dried crude purified extract prepared in Example 1 was dissolved in 1.0 ml of distilled water (HPLC grade) and subject to gel filtration chromatography according the condition disclosed in Table 1 in order to afford 20 fractions. Each fraction was added to protein dialysis membrane (Spectra/por 7, Spectrum Co. USA) and the membrane was dipped into a cylindrical glass flask containing 500 ml of distilled water (HPLC grade) to perform dialysis with stirring for 90 mins. After finishing desalting process with dialysis, the desalted solution present within the membrane was lyophilized for 3 days using by lyophilizer (FDCF-12012, Operon Co. Korea) and each purified fraction was collected to obtain 74 mg of purified extract of bee venom (yield: 74%) (designated as “HP-01G” hereinafter).

TABLE 1
Condition of Gel filtration analysis

| | |
|---|---|
| Pump | Knauer Smartline Pump 1000 (A50303) |
| Detector | Knauer S2600 UV detector |
| Column | GE Healthcare Superdex 75 16/600 PG |
| | (Preparative) |
| Flow rate | 0.9 ml/min |
| UV absorbance | UV 215 nm/UV 280 nm |
| Mobile phase | 50 mM Ammonium acetate with 0.5M |
| | NaCl (pH 4.0) |
| Sample | 100 mg of test sample was dissolved in 1 ml |
| | distilled water |
| Collection | Collected for 5 mins per test tube |

The component of HP-01 and HP-01G was analyzed using by HPLC according to the condition disclosed in Table 7 and the result was shown in Table 2.

As can be seen in table 2, it has been confirmed that the amount of phospholipase, melittin, apamine, histamine, dopamine, and adrenaline is 10.2%, 40.5%, 3.8%, 1.6%, 1.1% and 0.3% in HP-01 and 12.4%, 48.4%, 4.3%, 0.9%, 1.4% and 0.4% in HP-01G, respectively.

TABLE 2
The component ratio of main ingredients in HP-01 and HP-01G

| Component | HP-01 (%) | HP-01G (%) |
|---|---|---|
| Phospholipase A2 | 10.2 | 12.4 |
| Melittin | 40.5 | 48.4 |
| Apamin | 3.8 | 4.3 |
| Histamine | 1.6 | 0.9 |
| Dopamine | 1.1 | 1.4 |
| Noradrenaline | 0.3 | 0.4 |

### Example 3

### Preparation of Purified Extract of Bee Venom Using by Salting-Out Method

100 mg of the dried crude purified extract prepared in Example 1 was dissolved in 5.0 ml of distilled water (HPLC grade) to be adjusted to 20 mg/ml and the solution was subject to salting-in process by adding ammonium sulfate with stirring for 1 hour at room temperature to be 30% ammonium sulfate solution dropwisely. The solution was further stirred for 1 hour at room temperature and subjected to salting-out process by adding ammonium sulfate dropwisely to be 80% solution.

The solution was left alone for 2 hours at 0° C. to provide enough time to sufficient salting out process and centrifuged for 15 mins with the speed of 15,000 rpm by using ultra-speed centrifuges (Ultra 5.0, Hanil Science Medical Co. Ltd, Korea). The supernatant was collected and the precipitant was dissolved in 5 ml of distilled water (HPLC grade) in order that each one was subjected to be desalted and lyophilized to obtain 19 mg of purified extract of supernatant (designated as “HP-01AL” hereinafter) and 62 mg of purified extract of precipitant (designated as “HP-01AP” hereinafter) (total yield: 81%).

The component of HP-01AL and HP-01AP was analyzed using by HPLC according to the condition disclosed in Table 7 and the result was shown in Table 3.

As can be seen in Table 3, it has been confirmed that the amount of phospholipase, melittin, apamine, histamine, dopamine, and adrenaline is <0.1%, <0.1%, <0.1%, 8.4%, 3.1% and 1.2% in HP-01AL and 13.4%, 53.6%, 5.1%, <0.1%, <0.1%, and <0.1% in HP-01AP, respectively whereas those are 10.2%, 40.5%, 3.8%, 1.6%, 1.1% and 0.3% in HP-01.

TABLE 3
The component ratio of main ingredients in HP-01, HP-01AL and HP-01AP

| Component | HP-01 (%) | HP-01AL (%) | HP-01AP (%) |
|---|---|---|---|
| Phospholipase A2 | 10.2 | <0.1 | 13.4 |
| Melittin | 40.5 | <0.1 | 53.6 |
| Apamin | 3.8 | <0.1 | 5.1 |
| Histamine | 1.6 | 8.4 | <0.1 |
| Dopamine | 1.1 | 3.1 | <0.1 |
| Noradrenaline | 0.3 | 1.2 | <0.1 |

### Example 4

### Preparation of Purified Extract of Bee Venom Using by Solvent-Precipitation Method

100 mg of the dried crude purified extract prepared in Example 1 was dissolved in 2.5 ml of distilled water (HPLC grade) and ethanol having kept at −20° C. was added thereto to be adjusted to 10 ml of 75% (v/v) ethanol.

The solution was left alone for 2 hours at 0° C. to provide enough time to sufficient precipitation process and centrifuged for 15 mins with the speed of 15,000 rpm by using ultra-speed centrifuges (Ultra 5.0, Hanil Science Medical Co. Ltd, Korea). The supernatant and the precipitant was collected and each one was subjected to be desalted and lyophilized to obtain 13 mg of purified extract of supernatant (designated as “HP-01SL” hereinafter) and 69 mg of purified extract of precipitant (designated as “HP-01SP” hereinafter) (total yield: 82%).

The component of HP-01SL and HP-01SP was analyzed using by HPLC according to the condition disclosed in Table 7 and the result was shown in Table 4.

As can be seen in Table 4, it has been confirmed that the amount of phospholipase, melittin, apamine, histamine, dopamine, and adrenaline is <0.1%, <0.1%, <0.1%, 12.1%, 6.4% and 2.2% in HP-01SL and 7.8%, 56.4%, 5.8%, <0.1%, <0.1%, and <0.1% in HP-01SP, respectively whereas those are 10.2%, 40.5%, 3.8%, 1.6%, 1.1% and 0.3% in HP-01.

TABLE 4
The component ratio of main ingredients in HP-01, HP-01SL and HP-01SP

| Component | HP-01 (%) | HP-01SL (%) | HP-01SP (%) |
|---|---|---|---|
| Phospholipase A2 | 10.2 | <0.1 | 7.8 |
| Melittin | 40.5 | <0.1 | 56.4 |
| Apamin | 3.8 | <0.1 | 5.8 |
| Histamine | 1.6 | 12.1 | <0.1 |
| Dopamine | 1.1 | 6.4 | <0.1 |
| Noradrenaline | 0.3 | 2.2 | <0.1 |

### Example 5

### Preparation of Purified Extract of Bee Venom Using by Ultra Speed Centrifuges

100 mg of the dried crude purified extract prepared in Example 1 was dissolved in distilled water (HPLC grade) to be 10 ml and the sample was added to cartridge equipped with 50 kDa membrane filter (cartridge, Centrprep YM-50, Milipore Co. Ltd, USA). The sample was further centrifuged for 30 mins with the speed of 3,000 G by using ultra-speed centrifuges (Ultra 5.0, Hanil Science Medical Co. Ltd, Korea) to obtain two different fractions, i.e., high-molecular fraction having M. W. of more than 50 kDa (designated as “HP-02A50” hereinafter) and low-molecular fraction having M. W. of less than 50 kDa (designated as “HP-02B50” hereinafter).

The low-molecular fraction having M. W. of less than 50 kDa was added to cartridge equipped with 10 kDa membrane filter (cartridge, Centrprep YM-10, Milipore Co. Ltd, USA). The sample was further centrifuged for 30 mins with the speed of 3,000 G by using ultra-speed centrifuges (Ultra 5.0, Hanil Science Medical Co. Ltd, Korea) to obtain two different fractions, i.e., higher-molecular fraction having M. W. ranging from 10 kDa to 50 kDa (designated as “HP-03” hereinafter) and lower-molecular fraction having M. W. of less than 10 kDa (designated as “HP-04” hereinafter).

The collected fractions were lyophilized to obtain 10 mg of HP-02A50, 62 mg of HP-03, and 12 mg of HP-04, respectively (total yield: 84%).

The component of HP-02A50, HP-03 and HP-04 was analyzed using by HPLC according to the condition disclosed in Table 7 and the result was shown in Table 5.

As can be seen in Table 5, it has been confirmed that the amount of phospholipase, melittin, apamine, histamine, dopamine, and adrenaline is 76.2%, <0.1%, <0.1%, <0.1%, <0.1%, and <0.1% in HP-02A50; <0.1%, 43.2%, 6.2%, <0.1%, <0.1%, and <0.1%, in HP-03; and <0.1%, <0.1%, <0.1%, 12.8%, 8.1%, and 1.3% in HP-04, respectively whereas those are 10.2%, 40.5%, 3.8%, 1.6%, 1.1% and 0.3% in HP-01.

TABLE 5
The component ratio of main ingredients
in HP-01, HP-02A50, HP-03 and HP-04

| | HP-01 | HP-02A50 | HP-03 | HP-04 |
|---|---|---|---|---|
| Component | (%) | (%) | (%) | (%) |
| Phospholipase | 10.2 | 76.2 | <0.1 | <0.1 |
| A2 | | | | |
| Melittin | 40.5 | <0.1 | 43.2 | <0.1 |
| Apamin | 3.8 | <0.1 | 6.2 | <0.1 |
| Histamine | 1.6 | <0.1 | <0.1 | 12.8 |
| Dopamine | 1.1 | <0.1 | <0.1 | 8.1 |
| Noradrenaline | 0.3 | <0.1 | <0.1 | |

### Example 6

### Preparation of Purified Extract of Bee Venom Using by Dialysis Method

500 mg of the dried crude purified extract prepared in Example 1 weighed by electronic balance (CP225D, Sartorius, Germany) was dissolved in 2 ml of distilled water (HPLC grade). The sample was added to protein dialysis membrane (Spectra/por 7, Spectrum Co. USA) and the membrane was dipped into a cylindrical glass flask containing 200 ml of distilled water (HPLC grade) to perform dialysis with stirring for 90 mins. After finishing desalting process with dialysis, the desalted solution present within the membrane was lyophilized for 3 days using by lyophilizer (FDCF-12012, Operon Co. Korea) to obtain 440 mg of purified extract of bee venom (yield: 88%) (designated as “HP-05” hereinafter).

The component of HP-05 was analyzed using by HPLC according to the condition disclosed in Table 7 and the result was shown in Table 5.

As can be seen in Table 6, it has been confirmed that the amount of phospholipase, melittin, apamine, histamine, dopamine, and adrenaline is 10.2%, 40.5%, 3.8%, 1.6%, 1.1%, and 0.3% in HP-05 whereas those are 10.2%, 40.5%, 3.8%, 1.6%, 1.1% and 0.3% in HP-01.

TABLE 6
The component ratio of main
ingredients in HP-01, and HP-05

| Component | HP-01 (%) | HP-05 (%) |
|---|---|---|
| Phospholipase A2 | 10.2 | 11.1 |
| Melittin | 40.5 | 43.7 |
| Apamin | 3.8 | 4.1 |
| Histamine | 1.6 | <0.1 |
| Dopamine | 1.1 | <0.1 |
| Noradrenaline | 0.3 | <0.1 |

### Experimental Example 1

### Determination of the Amount of Melittin

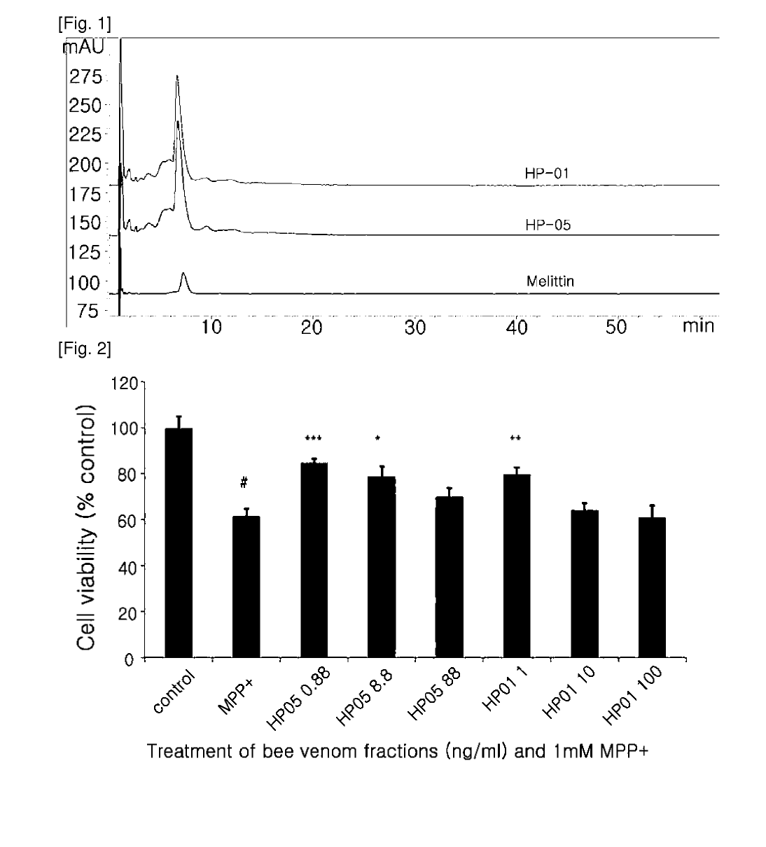

In order to determine the amount of melittin, a main ingredient of bee venom, the amount of melittin in bee venom was determined using by HPLC according to the condition disclosed in Table 7 and the result was analyzed using by following math FIG. 1.

MathFigure 1

AM (%)=AT (mg)×PS (%)/100×(PMSM)/(PMST)×100/ASS(mg) [Math.1]

AM: The amount of melittin (C₁₃₁H₂₂₉N₃₉O₃₁)

AT: the amount of standard

PS: the purity of standard

PMSM: the peak area of melittin in the sample

PMST: the peak area of melittin in the standard

ASS: the amount of sampling sample

TABLE 7
Condition of High Performance Liquid Chromatography

| | |
|---|---|
| Pump | Agilent 1200 Series, G1311A quat pump |
| Detector | Agilent 1200 Series, G1315D DAD |
| Column | μBondapak CN μm (3.9 × 150 mm) |
| Flow rate | 1.5 ml/min |
| UV absorbance | UV 214 nm |
| Mobile phase | Mixture solvent (water: Acetonitrile: |
| | TFA = 70:30:0.1) |
| Injection volume | 10 μl |

As can be seen in FIG. 1, the amount of melittin in HP-01 and HP-05 is 40.5% and 43.7%, respectively.

### Experimental Example 2

### Neuro-Protective Activity of Bee Venom Using by Neuroblastoma Cell Line

To assess the neuro-protective activity of the extract of bee venom, the neuro-protective activity of the extract of bee venom prepared in Examples was determined using by human neuroblastoma SH-SY5Y cells according to the modified procedure disclosed in the procedure (Yoshihisa Kitamura et al., Protective effects of the anti-Parkinsonnian drugs Talipexole and Pramipexole against 1-methyl-4-phenypyridinium-induced apoptotic death in human neuroblastoma SH-SY5Y cells, Mol. Pharmacol., 54 pp 1046-1054, 1998).

Human neuroblastoma SH-SY5Y cell line (Korea Cell Line Banks, Korea) was incubated in minimal essential medium containing 10% fetal bovine serum and 1% antibiotic-antimycotic solution at 37° C. under 6% CO₂ atmosphere.

The cell was inoculated into 48 well plates in the concentration of 1×10⁴ cells/well to incubate for 24 hours and various concentrations of HP-01 (0, 1, 10, 100 ng/ml) and HP-05 (0.88, 8.8, 88 ng/ml) were treated thereto to incubate for 3 hours.

1 mM MPP+(N-Methyl-4-phenylpyridinium ion; Sigma Co. USA) was treated thereto and incubated for 24 hours. 5 g of MTT solution [3-(4, 5-Dimethylthioazol-2-yl)-2,5-diphenyltetraazolium bromide, Sigma Co., USA] was dissolved in 1 L of PBS (Phosphate-buffered saline) to be 5 mg/ml and treated to the cells for 4 hours to be 0.05 mg/well to incubate for 4 hours at 37° C. After the incubation, the optical density of the samples was determined by using spectrometer (Spectramax Gemini XPS, Molecular device, USA) at 540 nm.

At the result, it has confirmed that both of HP-01 and HP-05 significantly inhibit the neuronal cell death and they have potent neuro-protective activity as can be seen in FIG. 2 (See, FIG. 2).

### Experimental Example 3

### Neuro-Protective Activity of Bee Venom Using by MPTP-Induced Degenerative Brain Disease Animal Model

To assess the neuro-protective activity of the extract of bee venom, the neuro-protective activity of the extract of bee venom prepared in Examples was determined using by MPTP-induced degenerative brain disease animal model according to the modified procedure disclosed in the procedure (Vernice Jackson-Lewis, Serge Przedborski, Protocol for the MPTP mouse mode of Parkinson's disease, Nature Protocols, 2(1) pp. 141-151, 2007).

12 weeks aged male C57BL/6 mouse weighing from 23 to 26 g (Samtako, Korea) was acclimated to environment for 1 week before use. The breeding room was controlled by automatic light system from 7:00 A.M. to 7:00 P.M, for 12 hours with adjusting the temperature to 24° C. and the mouse was freely let to access to water and feed.

In order to prepare Parkinson's disease-induced animal model, MPTP (1-methyl-4-phenyl-1,2,3,6-tetrahydropyridine, 30 mg/kg; Sigma Co., USA) in saline solution was intramuscularly injected into the mouse with 0.02 ml of 0.05% sample solution (HP-01, HP-05) for 5 days every 24 hours and similarly, saline solution was injected the mouse as a negative control. To determine whether the dopaminergic neuronal system caused by MPTP induction was damaged and whether bee venom treatment can protect the system or not, the change of TH (Tyrosine Hydroxylase) expression in striatum (ST) and substantia nigra (SN) was observed by using immune-histochemical staining method at the end of MPTP treatment.

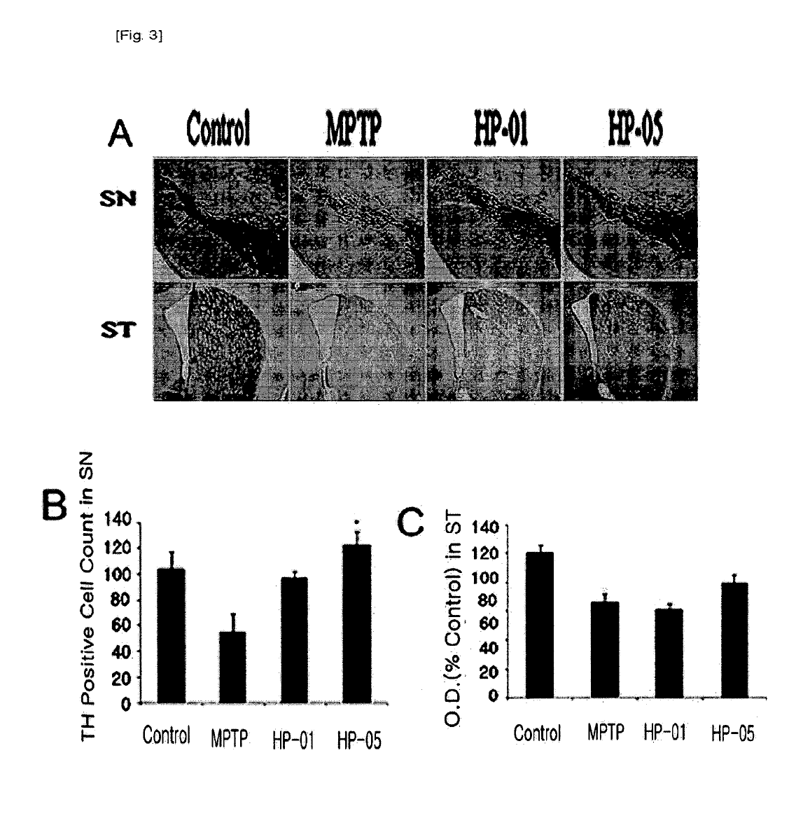

At the result, it has been confirmed that the groups treated with HP-01 and HP-05 showed potent neuro-protective activity comparing with that MPTP-sole treatment group (Parkinson's disease-induced group) while the number of TH positive group and neurons of the group intraperitoneally treated with MPTP were sharply reduced in striatum (ST) and substantia nigra (SN) as can be seen in FIG. 3 (See FIG. 3).

The number of TH positive cells present at substantia nigra (SN) in MPTP-treatment group was significantly reduced comparing with those in normal group however the group treated with HP-01 showed protective effect on dopaminergic neuron. In particular, the group treated with HP-05 showed most potent neuro-protective activity among them (p<0.05 vs MPTP), and the group treated with HP-05 showed increasing tendency of optical density (OD) value comparing with MPTP—treatment group. Accordingly, it has been confirmed that both of HP-01 and HP-05 can be useful in treatment of degenerative brain disease as well as Parkinson's disease.

### Experimental Example 4

### Inhibitory Effect of Bee Venom on the Microglial Activation Using by MPTP-Induced Degenerative Brain Disease Animal Model

To assess the inhibitory effect of the extract of bee venom on the microglial activation, the inhibitory effect of the extract of bee venom prepared in Examples was determined using by MPTP-induced degenerative brain disease animal model according to the modified procedure disclosed in the procedure (Vernice Jackson-Lewis, Serge Przedborski, Protocol for the MPTP mouse mode of Parkinson's disease, Nature Protocols, 2(1) pp. 141-151, 2007; Erwin bezard et al., A chronic MPTP model reproducing the slow evolution of Parkinson's disease; evolution of motor symptoms in the monkey, Brain Res., 766 pp. 107-112, 1997).

12 weeks aged male C57BL/6 mouse weighing from 23 to 26 g (Samtako, Korea) was acclimated to environment for 1 week before use. The breeding room was controlled by automatic light system from 7:00 A.M. to 7:00 P.M, for 12 hours with adjusting the temperature to 24° C. and the mouse was freely let to access to water and feed.

In order to prepare Parkinson's disease-induced animal model, MPTP (1-methyl-4-phenyl-1,2,3,6-tetrahydropyridine, 30 mg/kg; Sigma Co., USA) in saline solution was intramuscularly injected into the mouse with 0.02 ml of 0.05% sample solution (HP-01, HP-05) for 5 days every 24 hours and similarly, saline solution was injected the mouse as a negative control. To determine whether the dopaminergic neuronal system caused by MPTP induction was damaged and whether bee venom treatment can protect the system or not, the change of microglia cells stained with CD11B in striatum (ST) and substantia nigra (SN) was observed by using immune-histochemical staining method at the 7th day after the end of MPTP treatment.

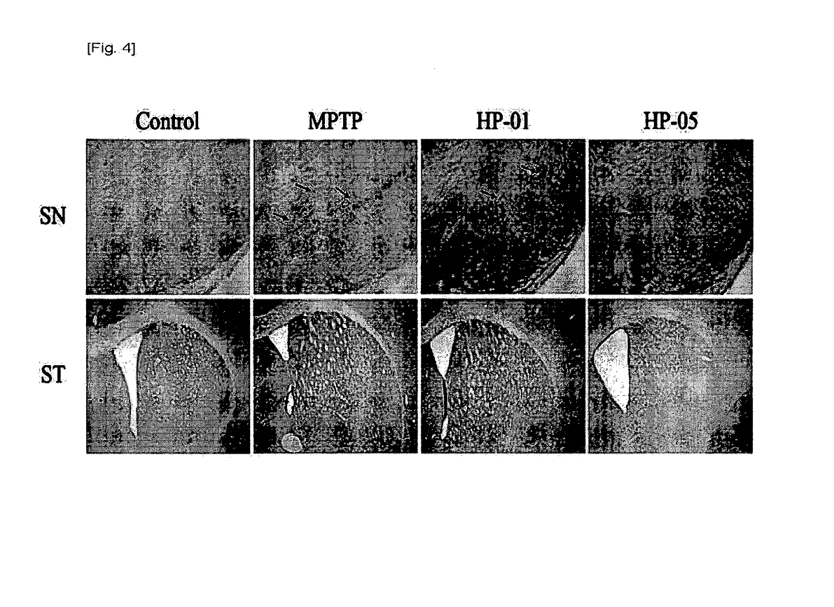

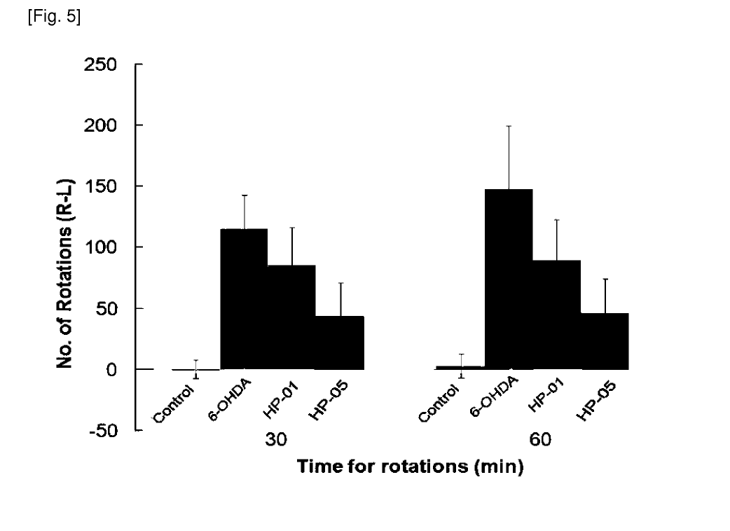

At the result, it has been confirmed that the number of microglia cell positive cell, TH positive cell and neuronal cells in the groups treated with HP-01 was significantly decreased and the group treated with HP-05 did not show any cells whereas many microglial cells were found in MPTP-treatment group as can be seen in FIG. 4 (See FIG. 4).

In order to examine the molecular biological mechanism of HP-01 and HP-05, the change of CD11B, a microglia marker, was determined and the MPTP-induced Parkinson's disease animal model showed increased activity of microglia cells while the group treated with HP-05 did not show the activity of microglia cells, of which result confirmed that HP-05 inhibit not only the inflammation response in brain but also the microglia cell activation.

### Experimental Example 5

### Effect of Bee Venom on the Change of Abnormal Circling Behavior Using by 6-OHDA-Induced Degenerative Brain Disease Animal Model

To assess the effect of the extract of bee venom on the change of abnormal circling behavior, the effect of the extract of bee venom prepared in Examples was determined using by 6-OHDA-induced degenerative brain disease animal model according to the modified procedure disclosed in the procedure (Andreas Schober, Classic toxin-induced animal models of Parkinson's disease: 6-OHDA and MPTP, Cell Tissue Res., 318 pp. 215-224, 2004).

Male Sprague Dawley rat weighing from 200 to 220 g (Samtako, Korea) was acclimated to environment for 1 week before use. The breeding room was controlled by automatic light system from 7:00 A.M. to 7:00 P.M, for 12 hours with adjusting the temperature to 24° C. and the rat was freely let to access to water and feed.

In order to prepare Parkinson's disease-induced animal model, pentobarbital injection (50 mg/kg, i.p.) was used as an anesthetics and 6-OHDA solution (25 μg/4 μl containing 0.01% L-ascorbic acid, Sigma Co., USA) was kept in the shaded refrigerator before use. 6-OHDA was injected into specific brain area (AP-0.7 mm, ML-2.6 mm, V-4.5 mm based on bregma) in a dose of 25 μg/4 μl at the speed of 1 μl/min by using 26-gauged Hamilton syringe.

After 6-OHDA was injected to the rat, HP-01 and HP-05 were administrated to the rat and 14 days after the treatment, and one week after the injection of 6-OHDA, apomorphine (0.5 mg/kg, subcutaneously, Sigma Co. USA) was injected to the rat. The spinning number of the rat was determined at every 30 and 60 mins using by automated rotometer chambers.

At the result, it has been confirmed that the groups treated with HP-01 showed reduced circling behavior comparing with 6-OHDA-treatment group, and especially, the group treated with HP-05 strongly inhibited abnormal circling behavior. Accordingly, it has been confirmed that both of HP-01 and HP-05 can be useful in treatment of degenerative brain disease as well as Parkinson's disease.

### Experimental Example 6

### Neuro-Protective Effect of Bee Venom Using by 6-OHDA-Induced Degenerative Brain Disease Animal Model

To assess the neuro-protective effect of the extract of bee venom, the neuro-protective effect of the extract of bee venom prepared in Examples was determined using by 6-OHDA-induced degenerative brain disease animal model according to the modified procedure disclosed in the procedure (Andreas Schober, Classic toxin-induced animal models of Parkinson's disease: 6-OHDA and MPTP, Cell Tissue Res., 318 pp. 215-224, 2004).

Male Sprague Dawley rat weighing from 200 to 220 g (Samtako, Korea) was acclimated to environment for 1 week before use. The breeding room was controlled by automatic light system from 7:00 A.M. to 7:00 P.M, for 12 hours with adjusting the temperature to 24° C. and the rat was freely let to access to water and feed.

In order to prepare Parkinson's disease-induced animal model, pentobarbital injection (50 mg/kg, i.p.) was used as an anesthetics and 6-OHDA solution (25 μg/4 μl containing 0.01% L-ascorbic acid, Sigma Co., USA) was kept in the shaded refrigerator before use. 6-OHDA was injected into specific brain area (AP-0.7 mm, ML-2.6 mm, V-4.5 mm based on bregma) in a dose of 25 μg/4 μl at the speed of 1 μl/min by using 26-gauged Hamilton syringe.

To determine whether the dopaminergic neuronal system caused by 6-OHDA induction was damaged and whether bee venom treatment can protect the system or not, the dopaminergic cells in striatum (ST) and substantia nigra (SN) was observed by using TH immune-histochemical staining method at the end of 6-OHDA treatment.

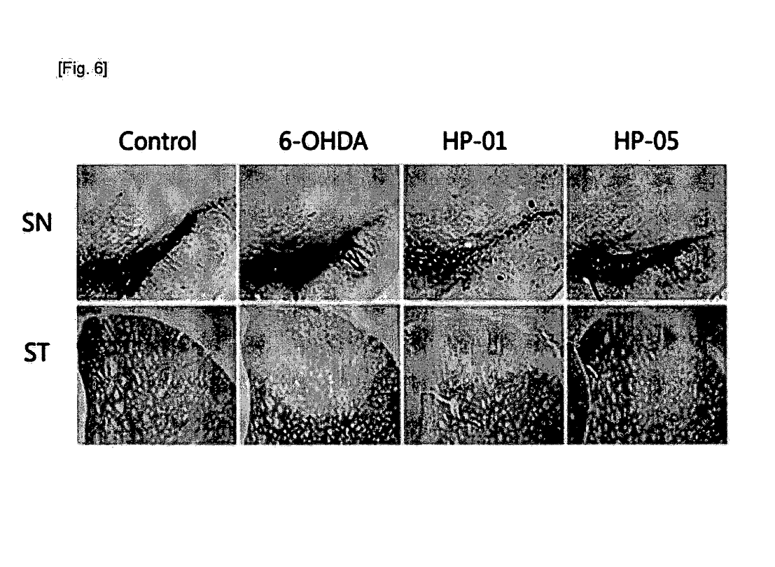

At the result, it has been confirmed that the groups treated with HP-01 and HP-05 showed potent neuro-protective activity comparing with that 6-OHDA treatment group with ST while the number of TH positive group and neurons of the group treated with 6-OHDA were sharply reduced in striatum (ST) and substantia nigra (SN) as can be seen in FIG. 6 (See FIG. 6).

The number of TH positive cells present at substantia nigra (SN) in 6-OHDA-treatment group was significantly reduced comparing with those in normal group however the group treated with HP-01 showed protective effect on dopaminergic neuron. In particular, the group treated with HP-05 showed more potent neuro-protective activity than that with HP-01 at the experiment in substantia nigra (SN) as well as striatum (ST). Accordingly, it has been confirmed that both of HP-01 and HP-05 can be useful in treatment of degenerative brain disease as well as Parkinson's disease.

### Experimental Example 7

### Clinical Test

To manifest the treating effect of the extract of bee venom on the patients suffering with Parkinson's disease, following clinical test was performed.

20 μg of HP-01 was intramuscularly injected to the specific acupuncture area on leg (point GB 34) of 10 volunteers consisting of 4 men and 6 women for 15 days once at the interval of three days. Before and after the test, the treating activity of the test sample was determined according to the general standard, i.e., UPDRS (United Parkinson's Disease rating Scale). The criteria of UPDRS was classified into 4 groups, i.e., UPDRS I (mention, behavior, mood: 1-4 contents, total score-16 points); UPDRS II (activities of daily living: 5-17 contents, total score-52 points); UPDRS III (motor examination: 18-31 contents, total score-108 points); and UPDRS IV (dyskinesia: 32-42 contents, total score-32 points). As the score is higher, the severity of the disorder is counted as being worsen.

The evaluation was determined through the interview between the volunteers and raters and before and after the test, the change of UPDRS was compared with each other through the evaluation.

At the result, although there exist individual difference between the volunteers, most of volunteers showed reduced total UPDRS value by about 10 points (See Table 8 and 9).

TABLE 8
Comparison of UPDRS score

| | | No. | | | | | | |
|---|---|---|---|---|---|---|---|---|
| | | 1 | 2 | 3 | 4 | 5 | 6 | 7 |
| Sex | | male | male | male | male | female | female | female |
| Age | | 68 | 68 | 67 | 62 | 70 | 69 | 65 |
| UPDRS | B* | 3 | 3 | 4 | 2 | 5 | 3 | 3 |
| I | A** | 2 | 2 | 2 | 2 | 3 | 2 | 1 |
| | D^{#} | 1 | 1 | 2 | 0 | 2 | 1 | 2 |
| UPDRS | B | 13 | 12 | 14 | 7 | 18 | 9 | 13 |
| II | A | 8 | 9 | 11 | 4 | 13 | 8 | 9 |
| | D | 5 | 3 | 3 | 3 | 5 | 1 | 4 |
| UPDRS | B | 14 | 17 | 19 | 11 | 23 | 7 | 16 |
| III | A | 9 | 14 | 14 | 8 | 15 | 5 | 14 |
| | D | 5 | 3 | 5 | 3 | 8 | 2 | 2 |
| UPDRS | B | 8 | 7 | 9 | 6 | 10 | 6 | 7 |
| IV | A | 5 | 6 | 7 | 5 | 4 | 3 | 2 |
| | D | 3 | 1 | 2 | 1 | 6 | 3 | 5 |
| UPDRS | B | 38 | 39 | 46 | 26 | 56 | 25 | 39 |
| total | A | 24 | 31 | 34 | 19 | 35 | 18 | 26 |
| | D | 14 | 8 | 12 | 7 | 21 | 7 | 13 |

B*: Before the test;
A**: After the test;
D^{#}: Difference between B* and A**

TABLE 9
Comparison of UPDRS score

| | | No. | | | |
|---|---|---|---|---|---|
| | | 8 | 9 | 10 | |
| | | Sex | | | |
| | | female | female | Female | |
| | | Age | | | |
| | | 64 | 62 | 59 | Mean |
| | | | | | 65.4 ± 3.596 |
| UPDRS I | B* | 4 | 3 | 2 | 3.2 ± 0.919 |
| | A** | 3 | 3 | 2 | 2.2 ± 0.632 |
| | D^{#} | 1 | 0 | 0 | 1.0 ± 0.816 |
| UPDRS II | B | 16 | 14 | 10 | 12.6 ± 3.273 |
| | A | 10 | 13 | 9 | 9.4 ± 2.633 |
| | D | 6 | 1 | 1 | 3.2 ± 1.814 |
| UPDRS III | B | 21 | 15 | 9 | 15.2 ± 5.138 |
| | A | 17 | 15 | 8 | 11.9 ± 4.012 |
| | D | 4 | 0 | 1 | 3.3 ± 2.312 |
| UPDRS IV | B | 9 | 8 | 4 | 7.4 ± 1.776 |
| | A | 8 | 7 | 2 | 4.9 ± 2.132 |
| | D | 1 | 1 | 2 | 2.5 ± 1.780 |
| UPDRS | B | 50 | 40 | 25 | 38.4 ± 10.637 |
| total | A | 38 | 38 | 21 | 28.4 ± 7.763 |
| | D | 12 | 2 | 4 | 10.0 ± 5.538 |

B*: Before the test;
A**: After the test;
D^{#}: Difference between B* and A**

The test result was analyzed according to one-way ANOVA analysis using by SPSS/PC+ package (p<0.05) and the Post Hoc Multiple Comparison was analyzed according to Duncan's multiple test. The value of each group was expressed as Means±SD.

Hereinafter, the formulating methods and kinds of excipients will be described, but the present invention is not limited to them. The representative preparation examples were described as follows.

Preparation of Powder

Dried powder of HP-01 300 mg

Lactose 100 mg

Talc 10 mg

Powder preparation was prepared by mixing above components and filling sealed package.

Preparation of Tablet

Dried powder of HP-02A50 50 mg

Corn Starch 100 mg

Lactose 100 mg

Magnesium Stearate 2 mg

Tablet preparation was prepared by mixing above components and entabletting.

Preparation of Capsule

Dried powder of HP-03 50 mg

Corn starch 100 mg

Lactose 100 mg

Magnesium Stearate 2 mg

Capsule preparation was prepared by mixing above components and filling gelatin capsule by conventional gelatin preparation method.

Preparation of Injection (1)

Dried powder of HP-05 2 mg

Distilled water for injectionoptimum amount

PH controller optimum amount

Injection preparation was prepared by dissolving active component, controlling pH to about 7.5 and then filling all the components in 2 ml ample and sterilizing by conventional injection preparation method.

Preparation of Injection (2)

Dried powder of HP-01 1 mg

Distilled water for injectionoptimum amount

PH controller optimum amount

Injection preparation was prepared by dissolving active component, controlling pH to about 7.5 and then filling all the components in 1 ml ample and sterilizing by conventional injection preparation method.

Preparation of Injection (3)

1 g of HP-01 was dissolved in 1000 ml of physiological saline solution. Various contaminants such as virus, germ and other impurities were removed using by anti-bacterial filter and then all the solution was added to 1 ml of vial. The solution was dried with lyophilization to be use as an injection preparation.

Preparation of Injection (4)

Dried powder of HP-05 0.88 mg

The above composition was filled in sterilized vial and was diluted with physiological saline solution for injection.

Preparation of Injection (5)

Dried powder of HP-05 0.88 mg

The above composition was dissolved in physiological saline solution for injection.

Various contaminants such as virus, germ and other impurities were removed using by anti-bacterial filter and then all the solution was added to 1 ml of vial. The solution was dried with lyophilization to be use as a injection preparation.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the purified extract of bee venom shows potent inhibitory effect on microglial cell activation and abnormal circling behavior using by animal model of degenerative brain disease as well as potent cell-protective activity therefore, it can be useful in treating and preventing the degenerative brain disease as a medicament.

1. A pharmaceutical composition comprising the purified extract of bee venom as an active ingredient for the treatment and prevention of degenerative brain disease by protecting neuronal cell.

2. The pharmaceutical composition according to claim 1 wherein said purified extract is the crude purified extract or the purified extract of bee venom such as honey bee, bumble bee, or sweat bee.

3. The pharmaceutical composition according to claim 2 wherein said purified extract is prepared by the procedure comprising the steps of: dissolving dried bee venom collected from honey bee in water, lower alcohols such as methanol, ethanol, or butanol, subjecting the solution to filtration to remove impurities from the solution, and drying the filtrates with lyophilization to obtain the inventive crude purified extract of bee venom.

4. The pharmaceutical composition according to claim 2 wherein said purified extract is prepared by the procedure comprising the steps of; dissolving the dried crude purified extract of bee venom prepared in the above-step in water, lower alcohols such as methanol, ethanol, or butanol, subjecting the solution to at least one purification process selected from salting out method, solvent precipitation method, and dialysis membrane filtration in order to performing centrifugation or dialysis, and drying the filtrates with lyophilization to obtain the inventive purified extract of bee venom.

5. The pharmaceutical composition according to claim 4 wherein said purified extract is prepared by the procedure comprising the steps of; dissolving dried bee venom collected from honey bee in water, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to dialysis membrane filtration using by membrane dialysis to collect the purified extract present in the membrane and drying the filtrates with lyophilization at 3^{rd} step to obtain the inventive purified extract of bee venom having more potent pharmacological effect than the other extract of bee venom.

6. The pharmaceutical composition according to claim 2 wherein said purified extract of bee venom comprises melittin in an amount of ranging from about 30% to 90% (w/w %) as an active ingredient.

7. The pharmaceutical composition according to claim 1 wherein said degenerative brain disease is Alzheimer type dementia, cerebrovascular type dementia, pick's disease, Creutzfeldt-jakob's disease, dementia caused by cephalic damage or Parkinson's disease.

8. The pharmaceutical composition according to claim 7 wherein said degenerative brain disease is Parkinson's disease.

9. The pharmaceutical composition according to claim 7 wherein said degenerative brain disease is Parkinson's disease caused by the hyper-activation of microglial cell.

10. The pharmaceutical composition according to claim 1 wherein said composition is a solid oral formulation, liquid oral formulation, topical preparation or parenteral dosage forms.

11. The pharmaceutical composition according to claim 10 wherein said composition is lyophilized preparation, emulsion, non-aqueous type injection, or aqueous type injection.

12. The pharmaceutical composition according to claim 11 wherein said composition is aqueous type injection intramuscularly, subcutaneously, or intracutaneously administered.

13. The pharmaceutical composition according to claim 12 wherein said composition is administered at the amount ranging from 8 microgram to 2 mg/day, of the inventive extract as set forth in claim 1.

14. A use of the purified extract of bee venom as set forth in claim 1 for the preparation of therapeutic agent for the treatment and prevention of degenerative brain disease by protecting neuronal cell in mammal including human.

15. A method of treating or preventing degenerative brain disease by protecting neuronal cell in mammal including human comprising administering an effective amount of the purified extract of bee venom as set forth in claim 1, together with a pharmaceutically acceptable carrier thereof to said mammal.

16. A method for preparing the purified extract of bee venom as set forth in claim 1 comprising the steps of; dissolving dried bee venom collected from honey bee in water, lower alcohols such as methanol, ethanol, or butanol, filtrating to remove impurities and drying with lyophilization to obtain the crude purified extract of bee venom comprising 10.2% phospholipase, 40.5% melittin, 3.8% apamine, 1.6% histamine, 1.1% dopamine and 0.3% adrenaline.

17. A method for preparing the purified extract of bee venom as set forth in claim 1 comprising the steps of; dissolving dried bee venom collected from honey bee in water, lower alcohols such as methanol, ethanol, or butanol, filtrating to remove impurities and drying with lyophilization to obtain the crude purified extract of bee venom as set for the in claim 16 at 1^{st} step; dissolving the crude purified extract prepared in step 1 in water, lower alcohols such as methanol, ethanol, or butanol, subjecting the solution to at least one purification process selected from salting out method, solvent precipitation method, and dialysis membrane filtration in order to performing centrifugation or dialysis, and drying the filtrates with lyophilization at 2^{nd} step to obtain the inventive purified extract of bee venom.

18. The method according to claim 17 wherein said method comprises the steps of; dissolving dried bee venom collected from honey bee in water, lower alcohols such as methanol, ethanol, or butanol, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to gel filtration chromatography and then protein dialysis membrane filtration using by membrane dialysis to perform salting out process at 3rd step; collecting the purified extract present in the membrane and drying the filtrates with lyophilization at 4th step to obtain the inventive purified extract of bee venom comprising 12.4% phospholipase, 48.4% melittin, 4.3% apamine, 0.9% histamine, 1.4% dopamine and 0.4% adrenaline.

19. The method according to claim 17 wherein said method comprises the steps of; dissolving dried bee venom collected from honey bee in water, lower alcohols such as methanol, ethanol, or butanol, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to salting-in process and then salt-out process using by using by salt such as ammonium sulfate to perform salting out process at 3rd step; centrifuging and lyophilizing the solution to obtain the supernatant at 4th step to obtain the inventive purified extract of bee venom in supernatant comprising <0.1% phospholipase, <0.1% melittin, <0.1% apamine, 8.4% histamine, 3.1% dopamine and 1.2% adrenaline and that in precipitant comprising 13.4% phospholipase, 53.6% melittin, 5.1% apamine, <0.1% histamine, <0.1% dopamine and <0.1% adrenaline.

20. The method according to claim 17 wherein said method comprises the steps of; dissolving dried bee venom collected from honey bee in water, lower alcohols such as methanol, ethanol, or butanol, filtrating to remove impurities and drying with lyophilization to obtain the crude purified extract of bee venom at 1^{st} step; dissolving the crude purified extract prepared in step 1 in the precipitation solvent selected from water, lower alcohols such as methanol, ethanol, or butanol or the mixture thereof, to occur precipitation at 2^{nd} step; subjecting the solution to centrifugation and lyophilizing the solution to obtain the supernatant at 3rd step to obtain the inventive purified extract of bee venom in supernatant comprising <0.1% phospholipase, <0.1% melittin, <0.1% apamine, 12.1% histamine, 6.4% dopamine and 2.2% adrenaline and that in precipitant comprising 7.8% phospholipase, 56.4% melittin, 5.8% apamine, <0.1% histamine, <0.1% dopamine and <0.1% adrenaline.

21. The method according to claim 17 wherein said method comprises the steps of; the steps of; dissolving dried bee venom collected from honey bee in water, lower alcohols such as methanol, ethanol, or butanol, filtrating to remove impurities and drying with lyophilization to obtain the crude purified extract of bee venom at 1^{st} step; dissolving the crude purified extract prepared in step 1 in the precipitation solvent selected from water, lower alcohols such as methanol, ethanol, or butanol or the mixture thereof, at 2nd step; subjecting the solution to ultra-centrifugation using by ultra-centrifuges quipped with 50 kDa membrane filter to obtain the purified extract of bee venom having high molecular weight of more than 50 kDa comprising 76.2% phospholipase, <0.1% melittin, <0.1% apamine, <0.1% histamine <0.1% dopamine and <0.1% adrenaline at 3rd step; centrifuging the fraction having low molecular weight of less than 50 kDa again using by ultra-centrifuges quipped with 10 kDa membrane filter to obtain the purified extract of bee venom having higher molecular weight ranging from 10 kDa to 50 kDa comprising <0.1% phospholipase, 43.2% melittin, 6.2% apamine, <0.1% histamine <0.1% dopamine and <0.1% adrenaline and that having lower molecular less than 10 kDa comprising <0.1% phospholipase, <0.1% melittin, <0.1% apamine, 12.8% histamine 8.1% dopamine and 1.3% adrenaline.

22. The method according to claim 17 wherein said method comprises the steps of; dissolving dried bee venom collected from honey bee in water, lower alcohols such as methanol, ethanol, or butanol, filtrating to remove impurities and drying with lyophilization to obtain the crude purified extract of bee venom at 1^{st} step; dissolving the crude purified extract prepared in step 1 in the solvent such as distilled water to obtain water soluble extract of bee venom at 2^{nd} step; subjecting the solution to dialysis process using by protein dialysis membrane at 3rd step; collecting the solution within the membrane and lyophilizing the solution to obtain the inventive purified extract of bee venom comprising 10.2% phospholipase, 40.5% melittin, 3.8% apamine, 1.6% histamine, 3.1% dopamine and 0.3% adrenaline.
